(19) United States
(12) Patent Application Publication
(10) Pub. No.: US 2014/0193370 A1
(43) Pub. Date: Jul. 10, 2014

(54) METHODS AND COMPOSITIONS FOR PROMOTING LEAN BODY MASS GROWTH

(71) Applicants: Delphine Tissot-Favre, Gollion (CH); Yuanlong Pan, Chesterfield, MO (US); George Wells, St. Louis, MO (US)

(72) Inventors: Delphine Tissot-Favre, Gollion (CH); Yuanlong Pan, Chesterfield, MO (US); George Wells, St. Louis, MO (US)

(73) Assignee: NESTEC SA, Vevey (CH)

(21) Appl. No.: 14/116,279

(22) PCT Filed: May 8, 2012
(86) PCT No.: PCT/US2012/036874

§ 371 Date: Feb. 6, 2014

Related U.S. Application Data
(60) Provisional application No. 61/518,704, filed on May 10, 2011.

(51) Int. Cl. A61K 31/16

(52) U.S. Cl. CPC A61K 31/16
USPC 424/93.4; 514/627; 564/208

(57) ABSTRACT

The invention provides methods for promoting lean body mass growth in an animal. The methods comprise administering OEA to the animals, preferably in amounts of from about 0.01 to about 1000 mg/kg/day.

This application claims priority to U.S. Provisional Application Ser. No. 61/518704 filed May 10, 2011, the disclosure of which is incorporated herein by this reference.

### BACKGROUND OF THE INVENTION

1. Field of the Invention

The invention relates generally to methods for promoting lean body mass growth and particularly to methods for using oleoylethanolamide (OEA) for promoting lean body mass growth in animals.

2. Description of Related Art

Lean body mass is essential for normal metabolism and normal physical activity. Therefore it is beneficial for animals, including humans and pets, to increase lean body mass to maintain health and quality of life. Unfortunately, when animals consume more calories than their maintenance requirement they usually convert the extra calories into body fat. Because of this conversion, chronic consumption of extra calories can lead to overweight and obese animals with higher fat to lean ratios. Obese and overweight animals have an increased risk of many chronic diseases including heart disease, diabetes, hypertension, stroke, dyslipidemia, certain types of cancer, apnea and osteoarthritis.

Current solutions for increasing lean to fat rations include reduced caloric intake, exercise and supplementation, for example with conjugated linoleic acid (CLA), isoflavones, and carnitine. Reducing caloric intake and regular exercise are difficult to practice. CLA and carnitine supplementation are quite expensive. Purified isoflavones are expensive and not all diets or foods contain soybean ingredients.

OEA is a naturally occurring lipid that regulates satiety and body weight. U.S. Pat. No. 7,423,066 describes the use of OEA for reducing body weight, modulating body lipid metabolism and reducing food intake. WO2004045307 describes food products and supplements comprising OEA for inducing satiety and decreasing weight.

Increasing lean body mass is critical for optimal metabolism and normal physical activity in animals. There is, therefore, a need for methods and compositions useful for promoting the health and wellness and improving the quality of life for animals by promoting lean body mass growth.

### SUMMARY OF THE INVENTION

It is, therefore, an object of the present invention to provide methods and compositions useful for promoting lean body mass growth in an animal.

It is another object of the invention to provide methods for promoting the health and wellness of animals.

It is another object of the invention to provide methods for extending the prime years of an animal's life.

It is a further object of the invention to provide compositions useful for promoting lean body mass growth in an animal.

One or more of these or other objects are achieved by administering OEA to animals in therapeutically effective amounts for promoting lean body mass growth. In general embodiments, OEA is administered to the animals in amounts of from about 0.01 to about 1000 milligrams per kilogram of body weight per day (g/kg/day) for as long as there is a need for such treatment.

Other and further objects, features, and advantages of the invention will be readily apparent to those skilled in the art.

### DETAILED DESCRIPTION OF THE INVENTION

### Definitions

The term “animal” means any animal that has a need for promoting lean body mass growth, including human, avian, bovine, canine, equine, feline, hicrine, lupine, murine, ovine, or porcine animals.

The term “companion animal” means domesticated animals such as cats, dogs, rabbits, guinea pigs, ferrets, hamsters, mice, gerbils, horses, cows, goats, sheep, donkeys, pigs, and the like.

The term “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “treating”, “treat”, and “treatment” embrace both preventative, i.e., prophylactic, and palliative treatment.

The terms “pharmaceutically acceptable” and “nutraceutically acceptable” indicates that the substance or composition must be compatible chemically and/or toxicologically, with the other ingredients comprising a formulation, and/or the mammal being treated therewith.

The term “health and/or wellness of an animal” means the complete physical, mental, and social well being of the animal, not merely the absence of disease or infirmity.

The term “extending the prime” means extending the number of years an animal lives a healthy life and not just extending the number of years an animal lives, e.g., an animal would be healthy in the prime of its life for a relatively longer time.

The term “in conjunction” means that OEA or other compounds or other compositions of the invention are administered to an animal (1) together in a food composition or (2) separately at the same or different frequency using the same or different administration routes at about the same time or periodically. “Periodically” means that OEA or other compounds or other compositions are administered on a schedule acceptable for specific compounds or compositions. “About the same time” generally means that OEA or other compounds or compositions are administered at the same time or within about 72 hours of each other.

The term “dietary supplement” means a product that is intended to be ingested in addition to a normal animal diet. Dietary supplements may be in any form, e.g., solid, liquid, gel, tablet, capsule, powder, and the like. Preferably they are provided in convenient dosage forms, e.g., in sachets. Dietary supplements can be provided in bulk consumer packages such as bulk powders, liquids, gels, or oils. Similarly such supplements can be provided in bulk quantities to be included in other food items such as snacks, treats, supplement bars, beverages, and the like.

The term “obese” means a medical condition in which excess body fat has accumulated to the extent that it may have an adverse effect on health. Body mass index (BMI), a measurement which compares weight and height, defines individuals as obese when it is greater than 30 kg/m². Dogs and cats are classified as obese when their body weight is 30% higher than their ideal body weight.

The term “overweight” means having more body fat than is optimally healthy. Individuals are considered overweight if their BMI is between 25 kg/m² and 30 kg/m². Overweight is considered to be pre-obese. Dogs and cats are classified as overweight when their body weight is 15 to 29% higher than their ideal body weight.

The term “food” or “food product” or “food composition” means a product or composition that is intended for ingestion by an animal, including a human, and provides nutrition to the animal.

The term “regular basis” means at least monthly dosing with OEA and more preferably weekly dosing. More frequent dosing or consumption, such as twice or three times weekly, is preferred in certain embodiments. Still more preferred are regimens that comprise at least once daily consumption, e.g., when OEA is a component of a food composition that is consumed at least once daily.

The term “single package” means that the components of a kit are physically associated in or with one or more containers and considered a unit for manufacture, distribution, sale, or use. Containers include, but are not limited to, bags, boxes, cartons, bottles, packages such as shrink wrap packages, stapled or otherwise affixed components, or combinations thereof. A single package may be containers of individual OEA and food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain instructions on how to use the kit or safety or technical information about one or more components of a kit.

The dosages expressed herein are in milligrams per kilogram of body weight per day (mg/kg/day) unless expressed otherwise.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a supplement”, “a method”, or “a food” includes a plurality of such “supplements”, “methods”, or “foods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### The Invention

In one aspect, the invention provides methods for promoting lean body mass growth in an animal. The methods comprise administering to the animal a therapeutically effective amount of OEA.

In a further aspect, the invention provides methods for promoting the health and wellness of an animal. The methods comprise administering to an animal a therapeutically effective amount of OEA.

In another aspect, the present invention provides methods for extending the prime years of an animal's life. The methods comprise administering OEA to an animal therapeutically effective amount of OEA.

In another aspect, the invention provides compositions comprising OEA in a therapeutically effective amount for promoting lean body mass growth.

The inventions are based upon the discovery that OEA has been found to promote lean body mass growth in animals. Specifically, dogs that were fed a weight gain diet with OEA had more gain in lean body mass and less body fat compared to dogs that were fed a weight gain diet without OEA.

Methods and compositions of the present invention may also be suitable for enhancing nutrient uptake; promoting lean body mass growth or anabolic muscle growth during chronic wasting, aging or infectious diseases; enhancing physical performance; enhancing a resistance training regime; preventing muscle breakdown or degradation during and after physical exertion; promoting recuperation from surgery faster by enhancing physiotherapy and preventing atrophy; and increasing muscle mass during childhood and adolescence.

In the methods of the invention, OEA is administered to an animal in amounts of from about 0.01 to about 1000 mg/kg/day, preferably from about 0.05 to about 500 mg/kg/day, most preferably from about 0.1 to about 250 mg/kg/day. In various embodiments, the OEA is administered in amounts of from about 0.5 to about 100 mg/kg/day.

In the methods of the invention, OEA is administered to an animal in amounts of from about 1-4000 mg, preferably from about 1 to about 2000 mg, most preferably from about 1 to about 1000 mg. In various embodiments, the OEA is administered in amounts of from about 1 to about 600 mg.

OEA can be administered to the animal in any suitable form using any suitable administration route. For example, OEA can be administered in an OEA composition, in a food composition, in a dietary supplement, in a pharmaceutical composition, in a nutraceutical composition, or as a medicament. Similarly, OEA can be administered using a variety of administration routes, including oral, intranasal, intravenous, intramuscular, intragastric, transpyloric, subcutaneous, rectal, and the like. Preferably, OEA is administered to an animal orally. Most preferably, OEA is administered orally to an animal as a dietary supplement or as an ingredient in a food composition.

In a preferred embodiment, OEA is administered to an animal as an ingredient in a food composition suitable for consumption by an animal, including humans and companion animals such as dogs and cats. Such compositions include complete foods intended to supply the necessary dietary requirements for an animal or food supplements such as animal treats. Examples of such food compositions include but are not limited to drinks, bars, frozen prepared foods and refrigerated prepared foods.

In various embodiments, food compositions such as pet food compositions or pet treat compositions comprise from about 5% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include beef, pork, lamb, equine, poultry, fish, and mixtures thereof.

The food compositions may further comprise from about 5% to about 40% fat. Examples of suitable fats include animal fats and vegetable fats. Preferably the fat source is an animal fat source such as tallow or poultry fat. Vegetable oils such as corn oil, sunflower oil, safflower oil, grape seed oil, soy bean oil, olive oil and other oils rich in monounsaturated and polyunsaturated fatty acids, may also be used.

The food compositions may further comprise from about 10% to about 60% carbohydrate. Examples of suitable carbohydrates include grains or cereals such as rice, corn, millet, sorghum, alfalfa, barley, soybeans, canola, oats, wheat, rye, triticale and mixtures thereof. The compositions may also optionally comprise other materials such as dried whey and other dairy by-products.

The moisture content for such food compositions varies depending on the nature of the food composition. The food compositions may be dry compositions (e.g., kibble), semi-moist compositions, wet compositions, liquid or semi-liquid composition, or any mixture thereof. In a preferred embodiment, the composition is a complete and nutritionally balanced pet food. In this embodiment, the pet food may be a “wet food”, “dry food”, or food of “intermediate moisture” content. “Wet food” describes pet food that is typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one preferred embodiment, the compositions have moisture content from about 5% to about 20%. Dry food products include a variety of foods of various moisture contents, such that they are relatively shelf-stable and resistant to microbial or fungal deterioration or contamination. Also preferred are dry food compositions that are extruded food products such as pet foods or snack foods for either humans or companion animals.

The food compositions may also comprise one or more fiber sources. The term “fiber” includes all sources of “bulk” in the food whether digestible or indigestible, soluble or insoluble, fermentable or nonfermentable. Preferred fibers are from plant sources such as marine plants but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefit provided by the present invention to the immune system of an animal.

In some embodiments, the ash content of the food composition ranges from less than 1% to about 15%, preferably from about 5% to about 10%.

In a preferred embodiment, the composition is a food composition comprising OEA and from about 15% to about 50% protein, from about 5% to about 40% fat, from about 5% to about 10% ash content, and having a moisture content of about 5% to about 20%. In other embodiments, the food composition further comprises prebiotics or probiotics as described herein.

When administered in a food composition, OEA comprises from about 0.001 to about 40% of the food composition, preferably from about 0.0025 to about 30%, more preferably from about 0.005 to about 20%. In various embodiments, food compositions comprise about 1%, 2%, 4%, 6%, 8%, 10%, 12%, 14%, 16%, 18%, 20%, 22%, 24%, 26%, 28%, 30%, 32%, 34%, 36%, 38%, or 40%.

In another embodiment, OEA is administered to an animal in a dietary supplement. The dietary supplement can have any suitable form such as a gravy, drinking water, beverage, yogurt, powder, granule, paste, suspension, chew, morsel, treat, snack, pellet, pill, capsule, tablet, sachet, or any other suitable delivery form. The dietary supplement can comprise OEA and optional compounds such as vitamins, preservatives, probiotics, prebiotics, and antioxidants. This permits the supplement to be administered to the animal in small amounts, or in the alternative, can be diluted before administration to an animal. The dietary supplement may require admixing with a food composition or with water or other diluent prior to administration to the animal. When administered in a dietary supplement, OEA comprises from about 0.001 to about 90% of the supplement, preferably from about 0.0025 to about 70%, more preferably from about 0.005 to about 60%.

In another embodiment, OEA is administered to an animal in a pharmaceutical or nutraceutical composition. The pharmaceutical composition comprises OEA and one or more pharmaceutically or nutraceutically acceptable carriers, diluents, or excipients. Generally, pharmaceutical compositions are prepared by admixing a compound or composition with excipients, buffers, binders, plasticizers, colorants, diluents, compressing agents, lubricants, flavorants, moistening agents, and the like, including other ingredients known to skilled artisans to be useful for producing pharmaceuticals and formulating compositions that are suitable for administration to an animal as pharmaceuticals. When administered in a pharmaceutical or nutraceutical composition, OEA comprises from about 0.001 to about 90% of the composition, preferably from about 0.0025 to about 70%, more preferably from about 0.005 to about 60%.

OEA can be administered to the animal on an as-needed, on an as-desired basis, or on a regular basis. A goal of administration on a regular basis is to provide the animal with a regular and consistent dose of OEA or the direct or indirect metabolites that result from such ingestion. Such regular and consistent dosing will tend to create constant blood levels of OEA and its direct or indirect metabolites. Thus, administration on a regular basis can be once monthly, once weekly, once daily, or more than once daily. Similarly, administration can be every other day, week, or month, every third day, week, or month, every fourth day, week, or month, and the like. Administration can be multiple times per day. When utilized as a supplement to ordinary dietetic requirements, OEA may be administered directly to the animal, e.g., orally or otherwise. OEA can alternatively be contacted with, or admixed with, daily feed or food, including a fluid, such as drinking water, or an intravenous connection for an animal that is receiving such treatment. Administration can also be carried out as part of a dietary regimen for an animal. For example, a dietary regimen may comprise causing the regular ingestion by the animal of OEA in an amount effective to accomplish the methods of the present invention.

According to the methods of the invention, OEA administration, including administration as part of a dietary regimen, can span a period ranging from parturition through the adult life of the animal. In various embodiments, the animal is a human or companion animal such as a dog or cat. In certain embodiments, the animal is a young or growing animal. In more some embodiments, the animal is an overweight or obese animal. In other embodiments administration begins, for example, on a regular or extended regular basis, when the animal has reached more than about 30%, 40%, or 50% of its projected or anticipated lifespan. In some embodiments, the animal has attained 40, 45, or 50% of its anticipated lifespan. In yet other embodiments, the animal is older having reached 60, 66, 70, 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan.

OEA is administered to an animal for a time required to accomplish one or more objectives of the invention, e.g., promoting lean body mass growth in an animal; improving the quality of life; and promoting the health and wellness in an animal. Preferably, OEA is administered to an animal on a regular basis.

In another aspect, the invention provides compositions comprising OEA in a therapeutically effective amount for one or more of promoting lean body mass growth in an animal; improving the quality of life in an animal; and promoting the health and wellness in an animal. The compositions contain OEA in amounts sufficient to administer OEA to an animal in amounts of from about 0.01 to about 1000 mg/kg/day, preferably from about 0.05 to about 500 mg/kg/day, more preferably from about 0.1 to about 250 mg/kg/day, most preferably from about 0.5 to about 100 mg/kg/day when the compositions are administered as anticipated or recommended for a particular composition. Typically, OEA comprises from about 0.001 to about 90% of a composition, preferably from about 0.005 to about 70%, more preferably from about 0.01 to about 60%. In various embodiments, food compositions comprise about 1%, 2%, 4%, 6%, 8%, 10%, 12%, 14%, 16%, 18%, 20%, 22%, 24%, 26%, 28%, 30%, 32%, 34%, 36%, 38%, 40%, 45%, 50%, 55%, 60%, 70%, or 80%.

OEA compositions such as food, dietary, pharmaceutical, and other compositions may further comprise one or more substances such as vitamins, minerals, probiotics, prebiotics, salts, and functional additives such as palatants, colorants, emulsifiers, and antimicrobial or other preservatives. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat soluble vitamins as A, D, E, and K. Inulin, amino acids, enzymes, coenzymes, and the like may be useful to include in various embodiments.

In various embodiments, the OEA compositions contain at least one of (1) one or more probiotics; (2) one or more inactivated probiotics; (3) one or more components of inactivated probiotics that promote health benefits similar to or the same as the probiotics, e.g., proteins, lipids, glycoproteins, and the like; (4) one or more prebiotics; and (5) combinations thereof. The probiotics or their components can be integrated into the OEA compositions (e.g., uniformly or non-uniformly distributed in the compositions) or applied to the OEA compositions (e.g., topically applied with or without a carrier). Such methods are known to skilled artisans, e.g., U.S. Pat. No. 5,968,569 and related patents.

Typical probiotics include, but are not limited to, probiotic strains selected from Lactobacilli, Bifidobacteria, or Enterococci, e.g., Lactobacillus reuteii, Lactobacillus acidophilus, Lactobacillus animalis, Lactobacillus ruminis, Lactobacillus johnsonii, Lactobacillus casei, Lactobacillus paracasei, Lactobacillus rhamnosus, Lactobacillus fermentum, and Bifidobacterium sp., Enterococcus faecium and Enterococcus sp. In some embodiments, the probiotic strain is selected from the group consisting of Lactobacillus reuteri (NCC2581; CNCM I-2448), Lactobacillus reuteri (NCC2592; CNCM I-2450), Lactobacillus rhamnosus (NCC2583; CNCM I-2449), Lactobacillus reuteri (NCC2603; CNCM I-2451), Lactobacillus reuteri (NCC2613; CNCM I-2452), Lactobacillus acidophilus (NCC2628; CNCM I-2453), Bifidobacterium adolescentis (e.g. NCC2627), Bifidobacterium sp. NCC2657 or Enterococcus faecium SF68 (NCIMB 10415). The OEA compositions contain probiotics in amounts sufficient to supply from about 10⁴ to about 10¹² cfu/animal/day, preferably from 10⁵ to about 10¹¹ cfu/animal/day, most preferably from 10⁷ to 10¹⁰ cfu/animal/day. When the probiotics are killed or inactivated, the amount of killed or inactivated probiotics or their components should produce a similar beneficial effect as the live microorganisms. Many such probiotics and their benefits are known to skilled artisans, e.g., EP1213970B1, EP1143806B1, U.S. Pat. No. 7,189,390, EP1482811B1, EP1296565B1, and U.S. Pat. No. 6,929,793. In a preferred embodiment, the probiotic is Enterococcus faecium SF68 (NCIMB 10415). In one embodiment, the probiotics are encapsulated in a carrier using methods and materials known to skilled artisans.

As stated, the OEA compositions may contain one or more prebiotics, e.g., fructo-oligosaccharides, gluco-oligosaccharides, galacto-oligosaccharides, isomalto-oligosaccharides, xylo-oligosaccharides, soybean oligosaccharides, lactosucrose, lactulose, and isomaltulose. In one embodiment, the prebiotic is chicory root, chicory root extract, inulin, or combinations thereof. Generally, prebiotics are administered in amounts sufficient to positively stimulate the healthy microflora in the gut and cause these “good” bacteria to reproduce. Typical amounts are from about one to about 10 grams per serving or from about 5% to about 40% of the recommended daily dietary fiber for an animal. The probiotics and prebiotics can be made part of the composition by any suitable means. Generally, the agents are mixed with the composition or applied to the surface of the composition, e.g., by sprinkling or spraying. When the agents are part of a kit, the agents can be admixed with other materials or in their own package. Typically, the food composition contains from about 0.1 to about 10% prebiotic, preferably from about 0.3 to about 7%, most preferably from about 0.5 to 5%, on a dry matter basis. The prebiotics can be integrated into the compositions using methods known to skilled artisans, e.g., U.S. Pat. No. 5,952,033.

A skilled artisan can determine the appropriate amount of OEA, food ingredients, vitamins, minerals, probiotics, prebiotics, antioxidants, or other ingredients to be use to make a particular composition to be administered to a particular animal. Such artisan can consider the animal's species, age, size, weight, health, and the like in determining how best to formulate a particular composition comprising OEA and other ingredients. Other factors that may be considered include the type of composition (e.g., pet food composition versus dietary supplement), the desired dosage of each component, the average consumption of specific types of compositions by different animals (e.g., based on species, body weight, activity/energy demands, and the like), and the manufacturing requirements for the composition.

In a further aspect, the invention provides kits suitable for administering OEA to animals. The kits comprise in separate containers in a single package or in separate containers in a virtual package, as appropriate for the kit component, OEA and one or more of (1) one or more ingredients suitable for consumption by an animal; (2) instructions for how to combine OEA and other kit components to produce a composition useful for promoting lean body mass growth; (3) instructions for how to use OEA for promoting lean body mass growth; (4) one or more probiotics; (5) one or more inactivated probiotics; (6) one or more components of inactivated probiotics that promote health benefits similar to or the same as the probiotics, e.g., proteins, lipids, glycoproteins, and the like; (7) one or more prebiotics; (8) a device for preparing or combining the kit components to produce a composition suitable for administration to an animal; and (9) a device for administering the combined or prepared kit components to an animal. In one embodiment, the kit comprises OEA and one or more ingredients suitable for consumption by an animal. In another embodiment, the kit comprises instructions for how to combine OEA and the ingredients to produce a composition useful for promoting lean body mass growth.

When the kit comprises a virtual package, the kit is limited to instructions in a virtual environment in combination with one or more physical kit components. The kit contains OEA and other components in amounts sufficient for promoting lean body mass growth. Typically, OEA and the other suitable kit components are admixed just prior to consumption by an animal. The kits may contain the kit components in any of various combinations and/or mixtures. In one embodiment, the kit contains a packet containing OEA and a container of food for consumption by an animal. The kit may contain additional items such as a device for mixing OEA and ingredients or a device for containing the admixture, e.g., a food bowl. In another embodiment, OEA is mixed with additional nutritional supplements such as vitamins and minerals that promote good health in an animal. The components are each provided in separate containers in a single package or in mixtures of various components in different packages. In preferred embodiments, the kits comprise OEA and one or more other ingredients suitable for consumption by an animal. Preferably such kits comprise instructions describing how to combine OEA with the other ingredients to form a food composition for consumption by the animal, generally by mixing OEA with the other ingredients or by applying OEA to the other ingredients, e.g., by sprinkling OEA on a food composition.

In a further aspect, the invention provides a means for communicating information about or instructions for one or more of (1) using OEA for promoting lean body mass growth; (2) contact information for consumers to use if they have a question regarding the methods and compositions of the invention; and (3) nutritional information about OEA. The communication means is useful for instructing on the benefits of using the invention and communicating the approved methods for administering OEA and food compositions containing OEA to an animal. The means comprises one or more of a physical or electronic document, digital storage media, optical storage media, audio presentation, audiovisual display, or visual display containing the information or instructions. Preferably, the means is selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, the invention provides methods for manufacturing a food composition comprising OEA and one or more other ingredients suitable for consumption by an animal, e.g., one or more of protein, fat, carbohydrate, fiber, vitamins, minerals, probiotics, prebiotics, and the like. The methods comprise admixing one or more ingredients suitable for consumption by an animal with OEA. Alternatively, the methods comprise applying OEA alone or in conjunction or combination with other ingredients onto the food composition, e.g., as a coating or topping. OEA can be added at any time during the manufacture and/or processing of the food composition. The composition can be made according to any method suitable in the art.

In another aspect, the invention provides a package useful for containing compositions of the present invention. The package comprises at least one material suitable for containing OEA and a label affixed to the package containing a word or words, picture, design, acronym, slogan, phrase, or other device, or combination thereof that indicates that the contents of the package contains OEA. In some embodiments, the label affixed to the package contains a word or words, picture, design, acronym, slogan, phrase, or other device, or combination thereof that indicates that the contents of the package contains OEA with beneficial properties relating to promoting lean body mass growth. Typically, such device comprises the words “preserves lean body mass during weight loss” or an equivalent expression printed on the package. Any package configuration and packaging material suitable for containing the composition is useful in the invention, e.g., bag, box, bottle, can, pouch, and the like manufactured from paper, plastic, foil, metal, and the like. In a preferred embodiment, the package further comprises the composition of the present invention. In a preferred embodiment, the package contains a food composition adapted for a particular animal such as a human, canine, or feline, as appropriate for the label, preferably a companion animal food composition for dogs or cats. In a preferred embodiment, the package is a can or pouch comprising a food composition of the invention. In various embodiments, the package further comprises at least one window that permit the package contents to be viewed without opening the package. In some embodiments, the window is a transparent portion of the packaging material. In others, the window is a missing portion of the packaging material.

In another aspect, the invention provides for use of OEA to prepare a medicament for one or more of promoting lean body mass growth; improving the quality of life; and promoting the health and wellness in an animal. Generally, medicaments are prepared by admixing a compound or composition, i.e., OEA or an OEA composition, with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

### EXAMPLES

The invention can be further illustrated by the following example, although it will be understood that this example is included merely for purposes of illustration and is not intended to limit the scope of the invention unless otherwise specifically indicated.

### Example 1

The physiological effects of regular oral OEA administration were investigated. OEA was provided as a supplement to dogs for four months to study the effect on body weight gain and body composition.

Eighteen adult dogs participated in the study. Males with less than 17.5% body fat and females with less than 20% body fat were selected. Based on maintenance energy requirements (MER,) body weight and percentage of body fat, the dogs were randomly assigned into two groups. The control group (N=9) received a standard dry adult dog food (control diet without any OEA). The test group (N=9) received a selected dose of OEA incorporated into the standard diet. The dogs were fed 25% more than their MER for eight months. During the first four months, they received their ration in one meal per day. From month four to eight, they received their ration equally divided in two meals per day. Body weight was recorded weekly and dual X-ray absorptiometry (DEXA) scans were performed at baseline and every two months. Food intake was recorded daily. There was no difference in food consumption between groups (i.e., similar caloric intake).

At the end of the eight month study, dogs fed the OEA-containing diet had less body fat (14.8 vs. 16.0%) and more gain in lean body mass (446.7 g vs. 236.9 g). For every kilogram of body weight gain, the dogs fed the OEA containing diet gained 22% more lean body mass than the dogs fed the control diet whereas the dog fed the control diet gained 56% more fat than the dogs fed the OEA containing diet (Table 1).

TABLE 1
Effect of Chronic Oral OEA on Lean and Fat Gain

| | Lean Gain | Fat Gain |
|---|---|---|
| | (g/kg body | (g/kg body |
| | weight gain) | weight gain) |
| Dogs Fed The Control Diet | 139 | 1333 |
| Dogs Fed The OEA Containing | 170 | 853 |
| Diet | | |

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims, the invention may be practiced otherwise than as specifically described.

What is claimed is:

1. A method for promoting lean body mass growth in an animal comprising administering to the animal a therapeutically effective amount of OEA.

2. The method of claim 1 wherein OEA is administered in amounts of from about 0.01 to about 1000 mg/kg/day.

3. The method of claim 1 wherein OEA is administered to the animal on a regular basis.

4. The method of claim 3 wherein OEA is administered in amount from about 1 to about 4000 mg.

5. The method of claim 1 wherein OEA is administered as a dietary supplement or a food composition.

6. The method of claim 5 wherein the composition is a food composition and OEA comprises from about 0.001 to about 10% of the food composition.

7. The method of claim 5 wherein the composition or dietary supplement further comprises one or more probiotics; inactivated probiotics; components of inactivated probiotics that promote health benefits similar to or the same as the probiotics; and one or more prebiotics.

8. The method of claim 1 wherein the animal is a human or a companion animal.

9. The method of claim 8 wherein the companion animal is a canine.

10. The method of claim 8 wherein the companion animal is a feline.

11. A composition comprising OEA in a therapeutically effective amount for promoting lean body mass growth.

12. The composition of claim 11 containing OEA in amounts sufficient to administer OEA to an animal in amounts to about from about 0.01 to about 1000 mg/kg/day.

13. A pharmaceutical or nutraceutical composition comprising OEA and one or more pharmaceutically or nutraceutically acceptable carrier, diluents or excipients

14. A package comprising at least one material suitable for containing OEA and a label affixed to the package containing a word or words, picture, design, acronym, slogan, phrase, or other device, or combination thereof that indicates that the contents of the package contains OEA.

15. (canceled)

16. (canceled)

17. (canceled)

18. (canceled)

19. (canceled)

20. (canceled)

21. (canceled)

22. (canceled)

23. (canceled)

24. (canceled)
